(19) United States
(12) Patent Application Publication
(10) Pub. No.: US 2022/0065874 A1
(43) Pub. Date: Mar. 3, 2022

(54) METHODS FOR THE IDENTIFICATION, ASSESSMENT, PREVENTION, AND TREATMENT OF NEUROLOGICAL DISORDERS AND DISEASES

(71) Applicants: THE JOHNS HOPKINS UNIVERSITY, Baltimore, MD (US); WAKE FOREST UNIVERSITY, Winston-Salem, NC (US)

(72) Inventors: Esther Oh, Baltimore, MD (US); Constantine Lyketsos, Baltimore, MD (US); Suzanne Craft, Winston-Salem, NC (US)

(21) Appl. No.: 17/013,974

(22) Filed: Sep. 8, 2020

Related U.S. Application Data
(63) Continuation of application No. 16/738,018, filed on Jan. 9, 2020, now abandoned.
Application No. 16/738,018 is a continuation of application No. 15/552,041, filed on Aug. 18, 2017, now abandoned.
(60) Provisional application No. 62/118,887, filed on Feb. 20, 2015.

(51) Int. Cl. G01N 33/68; G01N 33/48; A61K 38/48; C07K 16/18; G01N 33/66

(52) U.S. Cl. CPC G01N 33/6896; G01N 33/48; A61K 38/488; C07K 16/18; C12Y 304/23046; G01N 33/66; G01N 2800/50; G01N 2800/2814; G01N 2800/52; C07K 2317/76; G01N 2333/605; G01N 2333/62; G01N 2800/2821; G01N 2333/4709

(57) ABSTRACT

Described herein are methods of identifying a mammal having a neurological disease or disorder, such as AD or MCI, or at risk for developing a neurological disease or disorder, such as AD or MCI. Provided herein are also methods of monitoring the progression of a neurological disease or disorder in a patient or monitoring the effectiveness of therapeutic agent or treatment of a patient having a neurological disease or disorder.

### CROSS REFERENCE TO RELATED APPLICATIONS

This application is a Continuation of U.S. patent application Ser. No. 16/738,018, filed Jan. 9, 2020, which is a Continuation of U.S. patent application Ser. No. 15/552,041, filed Aug. 18, 2017, which is a 35 U.S.C. § 371 U.S. national entry of International Application PCT/US2016/018440, having an international filing date of Feb. 18, 2016, which claims the benefit of U.S. Provisional Application No. 62/118,887, filed Feb. 20, 2015, the content of each of the aforementioned applications is herein incorporated by reference in their entirety.

### GOVERNMENT SUPPORT

This invention was made with government support under grant numbers AG0337695, RR025006, AG043504-01, AG005146, and TR001079 awarded by the National Institutes of Health. The government has certain rights in the invention.

### BACKGROUND

Alzheimer's disease (AD) is the most common type of dementia, with an estimated 5.2 million sufferers in the U.S. (Alzheimer's Association, 2014). Despite an increased incidence and mortality, there is still no disease modifying treatment available. Due to a recent series of clinical trials with disappointing results, there is growing interest in interventions that target earlier stages of AD (Sperling, Jack, Aisen, 2011) such as the preclinical or mild cognitive impairment (MCI) stages. This shift to earlier stages of AD has made biomarkers an integral part of a clinical trial design, as they may be useful in identifying those who would benefit most from a potential therapeutic intervention.

One of the most widely studied biomarkers for AD is amyloid-beta (Aβ), thought to be an important protein in the pathogenic cascade of AD (Selkoe, 1999). Cerebrospinal fluid (CSF) and brain imaging measures of Aβ have been extensively studied and are now part of clinical trials. Although a blood-based biomarker would be even more widely applicable as it would be less invasive and less costly, most cross-sectional studies of plasma Aβ levels have not been able to show differences between individuals at various stages of AD compared to controls (Oh, Troncoso, Fangmark Tucker, 2008; Oh et al., 2010). In addition, the utility of plasma Aβ in earlier stages, such as MCI is less clear; as a recent systematic review found only one study supporting the utility of plasma Aβ in predicting who with MCI will later develop Alzheimer's or another dementias (Ritchie et al., 2014).

In order to overcome these limitations, efforts to improve the utility of plasma Aβ levels using different modulators have been investigated (Oh, Troncoso, Fangmark Tucker, 2008). These range from using insulin infusion in humans to change plasma and CSF levels (Watson et al., 2003; Kulstad et al., 2006a) to administration of anti-amyloid antibodies to induce efflux of Aβ into the periphery in transgenic (tg) animal models of AD (DeMattos et al., 2001). More recently, intraperitoneal glucose tolerance testing (IPGTT) was used to modulate the plasma Aβ levels in tg animal models of AD (Takeda et al., 2009) while oral glucose tolerance test (OGTT) was used to compare AD patients to those with non-AD dementias (Takeda et al., 2012). However, it is still unknown whether a modulator of Aβ plasma levels, such as OGTT, can be used to distinguish individuals in the earlier stages of AD from those with normal cognitive function. Described herein, in part, are methods using plasma amyloid-beta (Aβ) as a biomarker to assess individuals who have mild cognitive impairment (MCI), Alzheimer's disease (AD) and normal cognition, by modulating the plasma (amyloid-beta) Aβ level with an oral glucose tolerance test (OGTT). Such methods are based in part on assessing whether individuals with MCI/AD have different degrees of change in plasma Aβ 40 or 42 levels compared to cognitively normal controls in response to oral glucose loading.

### SUMMARY OF THE INVENTION

A blood-based biomarker would be more widely applicable, such as in the developing world where most future AD cases are anticipated, as it would be less invasive and less expensive compared to cerebrospinal (CSF) based or brain imaging biomarkers. However, most cross-sectional studies involving plasma Aβ have not been able to show differences between individuals in various stages of Alzheimer's disease (AD) compared to controls. Therefore, plasma Aβ would be an important non-invasive biomarker in assessing MCI/AD patients in the earlier stages who would be ideal candidates for therapies. Moreover, modulators of Aβ plasma levels may be useful in distinguishing individuals in different stages of AD from standard controls.

Provided herein are methods to enhance the utility of plasma amyloid-beta (Aβ) as a biomarker to assess individuals who have neurological disorders, such as mild cognitive impairment (MCI), Alzheimer's disease (AD), and normal cognition, by modulating the plasma Aβ level by administration of a simple sugar such as glucose, preferably by means of an oral glucose tolerance test (OGTT), and evaluating the effect of modulation on plasma Aβ level. One embodiment of the claimed invention measures the plasma Aβ levels before and after the administration of an oral glucose load in an OGTT paradigm and can differentiate mild cognitive impairment (MCI)/AD subjects from age matched cognitively normal controls.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 includes four panels identified as panels A, B, C, and D. FIG. 1 shows characteristic changes (Δ) in plasma Aβ 40 and 42 levels in normal controls (NC) compared to the MCI/AD participants. Each panel shows plasma Aβ levels in one representative individual. Panel A depicts Aβ 40 levels in a NC subject. Panel B depicts Aβ 40 levels in a MCI subject. Panel C depicts Aβ 42 levels in a NC subject. Panel D depicts Aβ 42 levels in a MCI subject.

FIG. 2 shows scatter diagrams of the changes (Δ) in plasma Aβ 40 and 42 levels after OGTT between cognitively normal controls and MCI/AD individuals. (Aβ40 and 42 “delta” are representative of the data from Table 2, and Ab 40 and Ab 42 “deltasens” are representative of the changes from Table 3).

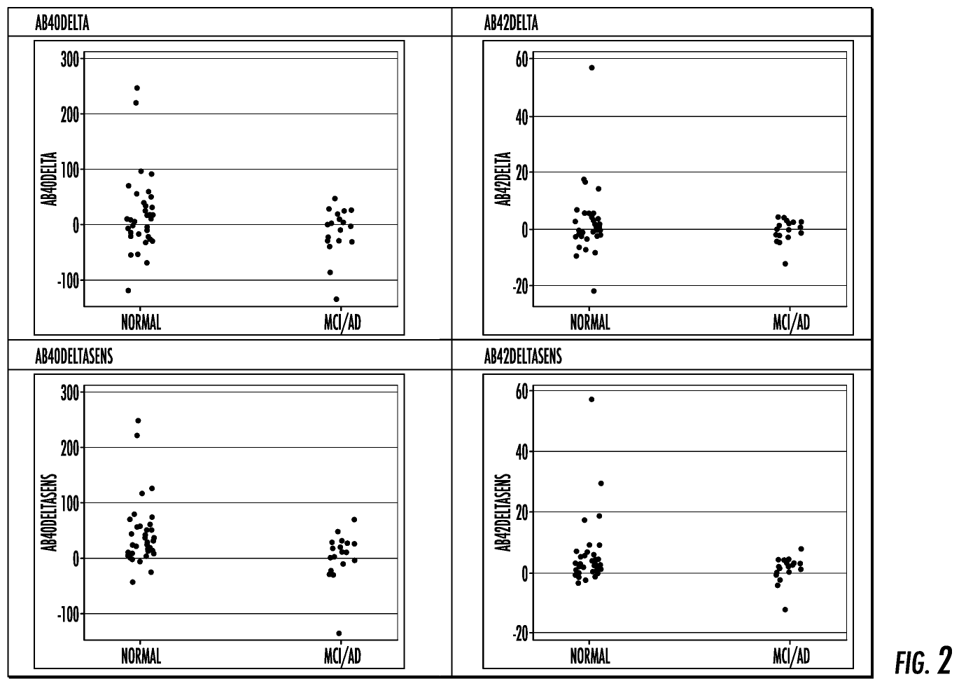

### DETAILED DESCRIPTION

### I. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

As used herein, the term “administering” a substance, such as a therapeutic entity to an animal or cell” is intended to refer to dispensing, delivering or applying the substance to the intended target. In terms of the therapeutic agent, the term “administering” is intended to refer to contacting or dispensing, delivering or applying the therapeutic agent to an animal by any suitable route for delivery of the therapeutic agent to the desired location in the animal, including delivery by either the parenteral or oral route, intramuscular injection, subcutaneous/intradermal injection, intravenous injection, buccal administration, transdermal delivery and administration by the intranasal or respiratory tract route.

As used herein, Amyloid beta (Aβ or Abeta) refers to the product which is formed after sequential cleavage of the amyloid precursor protein (APP), a transmembrane glycoprotein of undetermined function. APP can be cleaved by the proteolytic enzymes α-, β- and γ-secretase; Aβ protein is generated by successive action of the β and γ secretases. The γ secretase, which produces the C-terminal end of the Aβ peptide, cleaves within the transmembrane region of APP and can generate a number of isoforms of 36-43 amino acid residues in length. The most common isoforms are Aβ40 and Aβ42; the longer form is typically produced by cleavage that occurs in the endoplasmic reticulum, while the shorter form is produced by cleavage in the trans-Golgi network.

The term “biological sample” when used in reference to a diagnostic assay is intended to include tissues, cells and biological fluids isolated from a mammal, as well as tissues, cells and fluids present within a mammal, e.g., cerebrospinal fluid, spinal fluid, neural tissue, plasma, blood and components thereof.

As used herein, the terms “neurological diseases” or “neurological disorders” refers to a host of undesirable conditions affecting neurons or other cells in the brain of a subject and may be characterized, among others, by a progressive loss of neurons, neuronal cells, or loss of neuronal function. Examples of neurological diseases or disorders for which the current invention can be used preferably include, but are not limited to, Alzheimer's Disease (AD), Mild Cognitive Impairment (MCI), sporadic Alzheimer's disease, Lewy body disease, multiple system atrophy, dementia, senile dementia, Traumatic Brain Injury (TBI), Cerebral Amyloid Angiopathy (CAA), Frontotemporal Dementia (FTD), Normal Pressure Hydrocephalus (NPH), and Primary Progressive Aphasia (PPA).

A “patient” or “subject” or “mammal” refers to either a human or non-human animal. The term “treatment,” as used herein, is defined as the application or administration of a therapeutic agent to a patient, or application or administration of a therapeutic agent to an isolated tissue or cell line from a patient, who has a neurological disease or disorder, a symptom of a disease or disorder or a predisposition toward a disease or disorder, with the purpose of curing, healing, alleviating, relieving, altering, remedying, ameliorating, improving or affecting the disease or disorder, the symptoms of disease or disorder or the predisposition toward a disease or disorder. A therapeutic agent may be organic, inorganic or combinations thereof and includes, but is not limited to, biologics, antibodies, vaccines, polypeptides, peptides, peptidomimetics, ribozymes, cell or gene therapy, hormones, cytokines, tissue growth factors, nucleic acid molecules, aptamers, siRNA molecules, sense and antisense oligonucleotides drugs, small molecules, neutraceuticals, nano medicine, electroceuticals, medical devices and neural interfaces. In certain embodiments, treatment may halt, slow or reverse progression of the disease. In certain embodiments, treatment may include administering amyloid lowering agents. In other embodiments, treatment may include undergoing amyloid immunotherapy, such as using an antibody against the amyloid protein injected into a subject to remove the amyloid. In certain embodiments, treatment may comprise administering BACE (beta-site amyloid precursor protein (APP) cleaving enzyme) inhibitor, which would prevent further amyloid production by inhibiting cleavage of APP. In yet further embodiments, treatment may including administering agents that modulate the progression of neurological disease through alternative mechanismas of action including but not limited to Tau, glutamate, serotonin, neuronal nicotinic, RAGE, histidine, AChE, mitochondria, metabolic pathways and constituents including but not limited to insulin and HSD1.

As used herein, amyloid testing refers to clinical procedures typically performed by medical or health personnel in the examination of patients diagnosed with a neurological disorder, such as AD/MCI. Such amyloid testing may include, but not limited to, CSF collection, blood collection, or amyloid brain imaging.

The terms “comprise” and “comprising” are used in the inclusive, open sense, meaning that additional elements may be included.

The term “including” is used to mean “including but not limited to”. “Including” and “including but not limited to” are used interchangeably.

The term “standard control” is used to mean an accepted or approved example against which samples are judged or measured derived from one or more control subjects.

The term “oral glucose tolerance test” refers to an assay used to measure a subject's response to the sugar, glucose. Such assay may comprise obtaining a biological sample, such as blood, from a subject prior to having the subject ingest a certain amount of glucose. Thereafter, the subject ingests a liquid of a certain amount of glucose, such as 25 g to about 125 g, preferably from about 50 g to about 100 g and more preferably about 75 g. In certain embodiments, IV glucose tolerance test (IGTT) assay may be used as an alternative route for glucose administration. Subsequently, a biological sample, such as blood, is obtained from the subject at one or more time points from after administration of the glucose from about 1 minute to about 3 hours.

### II. Methods of Treatment

The present invention provides for prophylactic, diagnostic, prognostic, and therapeutic methods of identifying, treating, or preventing a neurological disease or disorder in a mammal, e.g., a human, at risk of (or susceptible to) a neurological disease or disorder, by subjecting the mammal to an oral glucose tolerance test (OGTT), obtaining a biological sample from the mammal, determining the levels, or change in levels with OGTT modulation, of one or more biomarkers such as Aβ, insulin, glucagon-like protein-1 (GLP-1), or combinations thereof, determining the level of expression or level of activity of said one or more biomarkers in a control, comparing the level of expression or level of activity of said one or more biomarkers in the mammal to a standard control, wherein modulation of the levels of the one or more biomarkers relative to the control indicates that the mammal is afflicted with a neurological disorder, or at risk of developing a neurological disorder. In certain embodiments, the modulation results in a increase or no change in Aβ 40, Aβ 42, or both, in said mammal after OGTT when compared to control. In some embodiment, a Δ Aβ 40 of about −140 pg/ml to about 60 pg/ml in the patient versus a Δ Aβ 40 of about −35 pg/ml to about 270 pg/ml in the control subject and a Δ Aβ 42 of about −15 pg/ml to about 6 pg/ml in the patient versus a Δ Aβ 42 of about −2 pg/ml to about 60 pg/ml in the control subject, may indicate that the patient has AD/MCI. By way of example, a Δ Aβ 40 of about −135.66 pg/ml to about 58.43 pg/ml in the patient versus a Δ Aβ 40 of about −32.15 pg/ml to about 263.51 pg/ml in the control subject and a Δ Aβ 42 of about −11.63 pg/ml to about 5.80 pg/ml in the patient versus a Δ Aβ 42 of about −1.38 pg/ml to about 57.21 pg/ml in the control subject, may indicate that the patient has AD/MCI. In certain embodiments, a increase or no change in Aβ 40, Aβ 42, or both of will indicate that the mammal has MCI, AD, or both. In other embodiments, a increase or no change in Aβ 40, Aβ 42, or both will indicate to the clinician that said mammal requires further invasive or non-invasive amyloid testing or MCI, AD, or MCI/AD treatment. In certain embodiments, the treatment will comprise administering to the subject one or more amyloid lowering agents, such that the neurological disease or disorder is treated or prevented. In some embodiments, which includes prophylactic and therapeutic methods, the one or more amyloid lowering agents is administered in a pharmaceutically acceptable formulation. Other treatments may include undergoing amyloid immunotherapy or treatment with BACE (beta-site amyloid precursor protein (APP) cleaving enzyme) inhibitor.

In other embodiments, the levels of GLP-1 or insulin will be concurrently measured using a combination of the diagnostic or prognostic assays described herein. In certain embodiments, the change in plasma GLP-1 levels in response to OGTT will be compared between MCI, AD, and cognitively normal controls, at a single cross section. In certain embodiments, patients with MCI, or AD, will have greater GLP-1 release in response to OGTT compared to cognitively normal controls. The level of GLP-1 release may be in the range of, but not limited to, about 0.6 pmol/L to about 51 pmol/L. In certain embodiments, the change in the plasma insulin levels in response to OGTT will be compared between MCI, AD, and cognitively normal controls, at a single cross section. In certain embodiments, patients with MCI, or AD, will have about 0 pmol/L to about 400 pmol/L of insulin in response to OGTT compared to cognitively normal controls. The level of insulin release may be in the range of about 0 pmol/L to about 400 pmol/L of insulin. In other embodiments, the levels of GLP-1 and insulin can be measured as a rate of release or an amount of release, for example, measure in units of pg/kg/min, ng/kg/min, ug/kg/min, or mg/kg/min.

A. Prophylactic, Diagnostic, Predictive, and Therapeutic Methods

The present invention also pertains to the field of predictive medicine in which diagnostic assays, prognostic assays, and monitoring of clinical trials are used for prognostic (predictive) purposes to thereby treat an individual prophylactically. Accordingly, one aspect of the present invention relates to diagnostic assays in the context of a biological sample (e.g., plasma, blood, serum, or fluid) to thereby determine whether or not an individual is afflicted with a neurological disease or disorder. The invention also provides for prognostic (or predictive) assays for determining whether an individual is at risk of developing a neurological disease or disorder.

In one aspect, the present invention provides a method for identifying a subject having a neurological disease or disorder, or at risk for developing a neurological disease or disorder. Subjects having a neurological disease or disorder, such as AD/MCI, may have significantly less change (Δ) in plasma compared to control subjects in both Aβ 40 and Aβ 42. In some embodiment, a Δ Aβ 40 of about −140 pg/ml to about 60 pg/ml in the patient versus a Δ Aβ 40 of about −35 pg/ml to about 270 pg/ml in the control subject and a Δ Aβ 42 of about −15 pg/ml to about 6 pg/ml in the patient versus a Δ Aβ 42 of about −2 pg/ml to about 60 pg/ml in the control subject, may indicate that the patient has AD/MCI. By way of example, a Δ Aβ 40 of about −135.66 pg/ml to about 58.43 pg/ml in the patient versus a Δ Aβ 40 of about −32.15 pg/ml to about 263.51 pg/ml in the control subject and a Δ Aβ 42 of about −11.63 pg/ml to about 5.80 pg/ml in the patient versus a Δ Aβ 42 of about −1.38 pg/ml to about 57.21 pg/ml in the control subject, may indicate that the patient has AD/MCI. Subjects at risk for a neurological disease or disorder can be identified by, for example, any or a combination of the diagnostic or prognostic assays described herein. In certain embodiments, normal subjects with plasma Aβ changes similar to the MCI/AD group would undergo more invasive or non-invasive amyloid testing. In such a scenario, administration of a prophylactic or therapeutic agent can occur prior to the manifestation of symptoms characteristic of a neurological disease or disorder, such that the neurological disease or disorder or symptom thereof, is prevented or, alternatively, delayed in its progression.

One particular embodiment includes a method for assessing whether a subject is afflicted with a specific neurological disease or disorder that may or may not currently have led to symptoms (e.g., MCI or asymptomatic AD), or is at risk of developing a neurological disease or disorder comprising detecting the expression or activity of the Aβ, GLP-1, insulin, or combinations thereof in a cell or tissue sample of a subject, wherein modulations of the expression or activity thereof indicates the presence of a neurological disease or disorder (with or without symptoms) or the risk of developing a neurological disease or disorder in the subject. In this embodiment, subject samples tested are, for example, plasma, cerebrospinal fluid, spinal fluid, or neural tissue.

Another aspect of the invention pertains to monitoring the influence of a glucose load, preferably by means of an OGTT on plasma Aβ in clinical trials. To determine whether a subject is afflicted with a neurological disease or disorder (with or without symptoms) has a risk of developing a neurological disease or disorder, a biological sample may be obtained from a patient immediately before and after being subjected to a glucose load, preferably by means of an OGTT and the levels of plasma Aβ (Aβ 40 or Aβ 42, or both), insulin, GLP-1, or combinations thereof, are detected over time, before and after the glucose load, preferably by means of an OGTT. A preferred agent for detecting the plasm Aβ, insulin, or GLP-1 levels may be an antibody or a labeled nucleic acid probe capable of hybridizing to the mRNA, genomic DNA, protein, or portions thereof, in a biological sample in vitro as well as in vivo. For example, in vitro techniques for detection of mRNA include Northern hybridizations and in situ hybridizations. In vitro techniques for detection of protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of protein include introducing into a subject a labeled antibody against the desired protein to be detected. For example, the antibody can be labeled with a detectable, such as a fluorescently labeled or radioactively-labeled, marker whose presence and location in the patient can be detected by standard imaging techniques. In certain embodiments, the Aβ levels are detected using MSD® Multi-spot Abeta Triplex Assay.

In another embodiment, the methods further involve subjecting a control patient to OGTT, obtaining a biological sample from the control patient immediately before and after the OGTT, detecting the control sample with a compound or agent capable of detecting Aβ, insulin, or GLP-1 protein, mRNA, or genomic DNA, such that the presence of the desired protein, mRNA or genomic DNA is detected in the biological sample, and comparing the presence, or change in level or expression or activity before and after OGTT, of the protein, mRNA or genomic DNA in the control sample with the presence of the protein, mRNA or genomic DNA in the patient sample. Such methods can be used to differentiate those who are “cognitively normal”, but already in the preclinical stages of AD. For example, cognitively normal with plasma Aβ changes (Δ) similar to the MCI/AD group will undergo more invasive or non-invasive amyloid testing.

B. Monitoring of Effects During Clinical Trials or Treatment

The present invention further provides methods for determining the effectiveness of a therapeutic regimen in treating or preventing a neurological disease or disorder or assessing risk of developing a neurological disease or disorder in a subject. For example, the effectiveness of a therapeutic treatment, prophylactic treatment, or therapeutic agent against AD/MCI can be monitored in clinical trials or other therapeutic regimen of subjects/patients using the present invention. In such clinical trials or therapeutic regimen, the degree of change (Δ) in Aβ 40, Aβ 42 levels before and after OGTT testing, or both can indicate the efficacy of a therapeutic agent. Small change (Δ) in plasma Aβ levels can serve as a marker, indicative of lack of physiological response of brain cells to the therapeutic agent, therapeutic treatment, or prophylactic treatment. This response state may be determined before, and at various points during treatment of the individual with the therapeutic agent, therapeutic treatment, or prophylactic treatment.

In other embodiments, the present invention provides a method for monitoring the effectiveness of treatment of a patient with an therapeutic agent, therapeutic treatment, or prophylactic treatment against AD/MCI or a patient at risk of developing AD/MCI, e.g. pre-clinical stage of AD; the methods including the steps of (i) obtaining a pre-administration sample from a patient prior to administration of the therapeutic agent; (ii) subjecting the patient to OGTT; (iii) detecting the levels of one or more biomarkers, such as plasma Aβ, insulin, GLP-1, or any combination thereof, in the pre-administration sample; (iv) obtaining one or more post-administration samples from the patient, typically in the first 10-20 minutes after the OGTT, occasionally as long as two hours later; (v) detecting the change in the levels of one or more biomarkers, such as plasma Aβ, insulin, GLP-1, or any combination thereof, in the post-administration samples; (vi) comparing the level of one of more biomarkers in step (iii) pre-administration sample with the level of one of more biomarkers in step (v) in the post-administration sample or samples; and (vii) altering the treatment of the subject accordingly. For example, a higher dose of the therapeutic agent may be desirable to increase the change of the Aβ levels to thereby increase the effectiveness of the therapeutic agent against MCI/AD. According to such an embodiment, change (Δ) of plasma Aβ levels may be used as an indicator of the effectiveness of a therapeutic agent or the appropriate dose of a therapeutic agent, even in the absence of an observable phenotypic response. In addition, change (Δ) of plasma Aβ levels may be used in choosing the most effective and appropriate treatment. Treatment choice may be informed by this assay at any time during disease progression from asymptomatic and prodromal through frank disease. This assay may also inform treatment choice for those who are at risk of developing a neurological disease. Treatments may be prophylactic, may provide symptomatic relief or may be disease modifying. Treatment may comprise disease management regimens to improve symptoms of memory loss and problems with thinking and reasoning, boost performance of chemicals in the brain, preserve cell to cell communication and brain function, preserve or improve neuronal bioenergetics, attenuate neuroinflammation and its sequelae or stop the underlying decline and death of brain cells.

The present invention additionally provides a kit comprising reagents and instructions for carrying out the method of any preceding claim.

All references cited herein are all incorporated by reference herein, in their entirety, whether specifically incorporated or not. All publications, patents, or patent applications cited herein are hereby expressly incorporated by reference for all purposes. In case of conflict, the definitions within the instant application govern.

Having now fully described this invention, it will be appreciated by those skilled in the art that the same can be performed within a wide range of equivalent parameters, concentrations, and conditions without departing from the spirit and scope of the invention and without undue experimentation.

The present description is further illustrated by the following examples, which should not be construed as limiting in any way.

### EXAMPLES

### Brief Summary:

Background: Plasma levels of amyloid-beta (Aβ) do not correlate well with different stages of Alzheimer's disease (AD) in cross-sectional studies.

Methods: 57 participants (18 with AD/MCI and 39 cognitively normal controls) underwent oral glucose tolerance testing (OGTT). Blood samples were obtained over a 2 hour time period. Plasma Aβ40 and Aβ42 levels were measured, and changes in plasma levels of Aβ40 and Aβ42 from either baseline or 5 minutes to the 10 minute time point were measured.

Results: Compared to normal controls, subjects with AD/MCI had significantly less change (Δ) in plasma levels for both Aβ 40 (−3.13 pg/ml vs. 41.34 pg/ml; p=0.002) and Aβ 42 (0.15 pg/ml vs. 5.64 pg/ml; p=0.004).

Conclusion: Oral glucose tolerance testing is potentially useful in distinguishing aging individuals who are in different stages of AD.

### Methods:

### Participants

This study was approved by the Johns Hopkins Institutional Review Board, and conducted at the Institute for Clinical and Translational Research (ICTR) Bayview Clinical Research Unit (Bayview CRU). Written informed consent was obtained from all subjects.

The study comprised 57 individuals, two with AD, 16 with MCI (MCI), and 39 with normal cognition (Table 1). AD and MCI participants were combined in the analysis (exclusion of the AD subjects did not change the results). Subjects with AD met probable AD criteria by NINCDS/ADRDA (National Institute of Neurological and Communicative Disorders and Stroke and the Alzheimer's Disorder and Related Disorder Association). MCI participants had a memory complaint corroborated by an informant, MCI documented in medical or research records, no or minimal impairment in activities of daily living (ADLs), and a Clinical Dementia Rating (CDR) of 0.5. Cognitively normal controls (NC) had no reported memory impairments by history, a CDR of 0.0, and MMSE ≥26 or 3-MS (Modified MMSE) ≥86. Subjects were excluded if they had significant neurologic disease such as stroke, Parkinson's disease, multiple sclerosis, severe head injury with loss of consciousness >30 min or permanent neurologic sequelae, liver dysfunction, renal dysfunction, significant cardiac disease, history of diabetes or treatment for diabetes.

### Procedures

Subjects were asked to fast for 12 hours prior to a single early morning study visit. Blood was drawn to obtain baseline measures prior to drinking a solution containing 75 g of glucose. Blood samples were then obtained 5, 10, 15, 30, 60, 90 and 120 minutes after drinking the solution.

Blood was collected in EDTA polypropylene tubes for plasma, and centrifuged immediately after each collection at 2200 rpm for 15 minutes at 4° C. GLP-1 samples were collected in pre-chilled tubes containing EDTA, peptidase inhibitors [aprotinin (trasylol) and DPP-4 inhibitors]. Plasma was divided into 0.25 ml aliquots, and stored at −80° C. until analysis.

ELISA Aβ40 and Aβ42 levels were measured in plasma (Oh et al., 2010) using the MSD® Multi-spot Abeta Triplex Assay (Meso Scale Discovery, Gaithersburg, Md.), procedure of the Alzheimer's Disease Cooperative Study (ADCS) Biomarker Core (Donohue et al., 2014). Previously unthawed aliquots were analyzed after thawing. All samples were run in duplicate, and internal standards were used to control for plate-to-plate variation. Aβ 40 and 42 values +/−SEM are reported in this article.

### Statistics

Baseline comparisons were made using two-sample t-tests with Satterthwaite's approximation for degrees of freedom. Aβ 40 and Aβ42 (Δ) values were calculated as the difference between the value at ten minutes and the maximum value occurring prior to ten minutes (at either 0 or 5 minutes). The trapezoidal rule was used to calculate integrated responses or Area Under the Curve (AUC) over 0-120 minutes for GLP-1. All analyses were conducted using STATA (StataCorp LP, College Station, Tex.).

### Results:

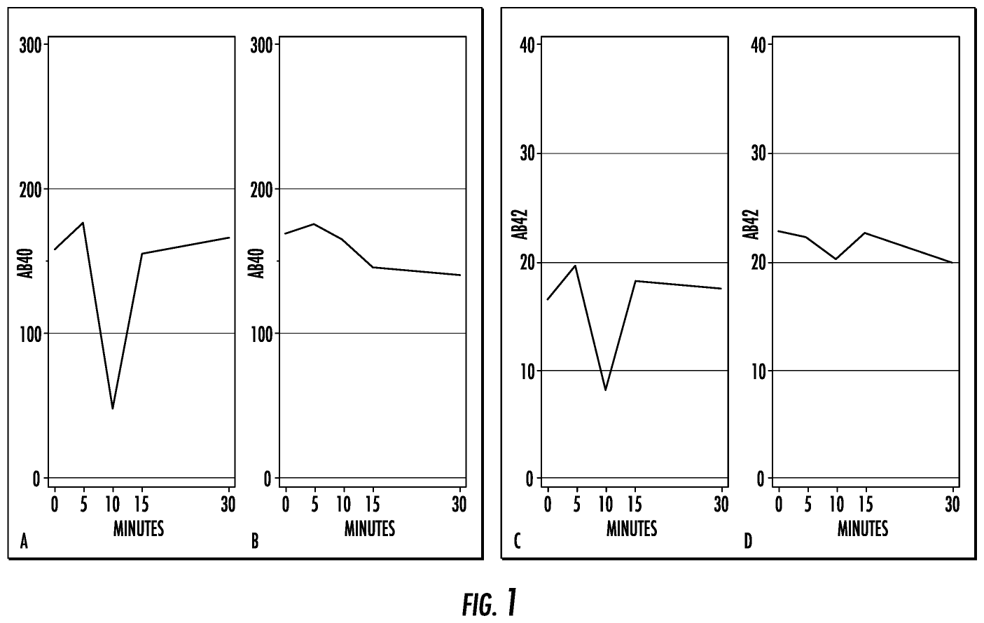

At baseline, no significant between group differences were observed in age, sex, education, fasting glucose, baseline plasma Aβ40 and 42 levels and Aβ 42/40 ratios (Table 1). We calculated the change (Δ) in plasma Aβ as the higher level of plasma Aβ from either 0 (baseline) or 5 minutes after ingestion of oral glucose solution to the 10 minute time point after ingestion. Subjects with AD/MCI had significantly less change(Δ) in plasma Aβ levels compared to controls in both Aβ 40 (−3.13 (40.93) pg/ml vs. 41.34 (57.16) pg/ml; p=0.002) and Aβ42 (−0.15 (3.77) pg/ml vs. 5.64 (10.65) pg/ml; p=0.004). Characteristic changes (Δ) in plasma Aβ40 and 42 levels are shown in FIG. 1. We also performed sensitivity and adjusted analyses. 9 subjects had well documented history of depression. Excluding these individuals did not change the differences significantly, with subjects with AD/MCI having less change (Δ) in plasma Aβ40 levels (−3.14 (40.93) pg/ml vs. 41.73 (60.99) pg/ml; p=0.004) and in Aβ42 levels (−0.15 (3.77) pg/ml vs. 6.38 (11.87) pg/ml; p=0.008). Although individuals with prior history of diabetes were excluded from the study, there were two subjects whose glucose levels at baseline (fasting) and 2 hours after OGTT met the American Diabetes Association criteria for Type II diabetes on the day of testing. We performed a sensitivity analysis excluding these individuals, and the magnitude of change (Δ) and the inference did not change with Aβ40 (−3.14 (40.93) pg/ml vs. 42.64 (57.55) pg/ml; p=0.001) or with Aβ42 (−0.15 (3.77) pg/ml vs. 5.75 (10.91) pg/ml; p=0.005). In separate logistic regressions of change (Δ) on diagnosis category, the unadjusted OR for Aβ40(Δ) was 0.97 (95% CI 0.94, 0.99; p=0.01) and for Aβ42(Δ) was 0.74 (95% CI 0.57, 0.96; p=0.02) which means that there is 3% less risk of being in the MCI/AD group for every 1 pg/ml difference in Aβ 40(Δ) and 26% less risk for every 1 pg/ml difference in Aβ 42(Δ). After adjusting for age and BMI, both odds ratios remained relatively unchanged and statistically significant; the OR for Aβ40(Δ) was 0.97 (95% CI 0.94, 0.99; p=0.008) and for Aβ42(Δ) was 0.73 (95% CI 0.56, 0.95; p=0.02). Subjects with AD/MCI had significantly greater GLP-1 response to OGTT compared to controls [234.76 (SD 123.16) vs. 154.58 (SD 88.63), p=0.01].

TABLE 1
Baseline Characteristics

| | | |
|---|---|---|
| Normal | MCI/AD | |
| (N = 39) | (N = 18) | |
| Mean (SD) | Mean (SD) | p-value |

Demographics

| | | | |
|---|---|---|---|
| Age (years) | 68.2 (6.98) | 70.6 (7.31) | 0.25 |
| Sex (M) (%) | 51.3% | 44.4% | 0.64 |
| Education (years) | 15.62 (2.37) | 15.28 (3.48) | 0.71 |
| MMSE | 29.3 (1.41) | 27.7 (2.27) | 0.01 |
| BMI | 28.23 (4.67) | 26.94 (4.07) | 0.28 |

Laboratory Values

| | | | |
|---|---|---|---|
| Fasting glucose mg/dl | 94.18 (15.48) | 91.22 (13.31) | 0.49 |
| Amy1oid-β (40) pg/ml | 192.37 (73.79) | 180.11 (75.10) | 0.57 |
| Amy1oid-β (42) pg/ml | 24.73 (23.77) | 17.85 (8.02) | 0.11 |
| Amy1oid-β 42/40 ratio | 0.18 (0.39) | 0.11 (0.04) | 0.27 |
| Δ Amy1oid-β (40) | 41.34 (57.16) | −3.14 (40.93) | 0.002 |
| pg/ml | | | |
| Δ Amy1oid-β (42) | 5.64 (10.65) | −0.15 (3.77) | 0.004 |
| pg/ml | | | |
| GLP-1 (AUC)^{a} | 154.58 (88.63) | 234.76 (123.16)^{b} | 0.01 |

^{a}Glucagon like peptide-1 (GLP-1), Area Under the Curve (AUC);
^{b}N = 21 subjects in this group

Conclusion

These findings suggest that individuals with MCI/AD have different degrees of change (Δ) in plasma Aβ 40 and 42 levels compared to cognitively normal controls within the first ten minutes of an oral glucose load. Although OGTT has been used previously as a modulator of plasma Aβ (Takeda et al., 2012), this study differs in that the focus is on comparing individuals with MCI or in the earlier stages of AD to cognitively normal controls. Takeda et al. focused on comparing individuals with fairly advanced AD to those with non-AD dementias, whose overall average mini-mental state examination (MMSE) scores ranged from 11-12 (Takeda et al., 2012). In addition, the present invention's finding shows greater decline in plasma Aβ 40 and 42 levels from baseline to 10 minutes in cognitive normal controls compared to MCI/AD individuals, not evident in the previous study, which examined plasma Aβ levels over a 2 hour time period, but did not include the 5 or 10 minute time points (Takeda et al., 2012).

At this time, the mechanism explaining these differences in the change in plasma Aβ level is unclear. It is possible that OGTT modulated plasma Aβ levels by increasing insulin secretion, as insulin is known to increase the level of plasma Aβ 42 in AD (Kulstad et al., 2006a). However, insulin level does not peak until 60-120 minutes after an OGTT (Meier et al., 2007), while the change in plasma Aβ levels occurred in the first 10 minutes after administration of glucose loading.

Another possible mechanism involves glucagon-like protein-1 (GLP-1), a gastrointestinal hormone derived from post-translational modification of the proglucagon gene (Hoist, 2007). This is produced in the L cells of the distal small intestine (Hoist, 2007), and secreted in response to a meal or after an oral glucose challenge. GLP-1 may be involved in hepatic clearance of Aβ. After production in intestinal cells, GLP-1 is transported to the liver via the portal vein (Dardevet et al., 2005), also thought to be the primary route of clearance for Aβ (Kulstad et al., 2006b). GLP-1 is also thought to play a role in amyloid precursor protein (APP) and Aβ regulation. In vitro experiments involving treatment of PC 12 cells with the GLP-1 and GLP-1 analogs exendin-4 and exendin-4-WOT reported significant decreases in intracellular levels of APP, a precursor protein of Aβ (Perry et al., 2003). While the mechanism remains speculative, both insulin and GLP-1 levels after OGTT will be examined in the future studies to further delineate their role.

Other mechanisms include but are not limited to incretins, GIP and lipids.

In summary, these study suggests that oral glucose loading as a plasma Aβ level modulator can “unmask” the differences between individuals with MCI/AD versus normal controls. One way this method might be utilized is to complement other existing biomarkers. For example, individuals with normal like drops in Aβ levels might not be good candidates for further amyloid oriented investigation via CSF collection or amyloid brain imaging in clinical trials—or vice-versa. In addition, this method might differentiate those who are “cognitively normal,” but already be in the preclinical stages of AD. In the latter case, normals with plasma Aβ changes similar to the MCI/AD group would undergo more invasive or non-invasive amyloid testing. Both these scenaria would reduce costs for AD clinical trials, but more importantly, spare individuals less likely to have AD pathology from undergoing unnecessary tests. This would be especially applicable in the developing world where most future AD cases are anticipated, but where resources are limited. OGTT has a distinct advantage as a safe, non-invasive, cost-effective, and widely available biomarker that is already being used in clinical settings world-wide.

### REFERENCES

1. Alzheimer's Association. 2014. 2014 Alzheimer's disease facts and figures. Alzheimers Dement. 10: e47-92. 51552526014000624 [pii].
2. Dardevet D, Moore M C, DiCostanzo C A, Farmer B, Neal D W, Snead W, Lautz M, Cherrington A D. 2005. Insulin secretion-independent effects of GLP-1 on canine liver glucose metabolism do not involve portal vein GLP-1 receptors. Am. J. Physiol. Gastrointest. Liver Physiol. 289: G806-14. 10.1152/ajpgi.00121.2005.
3. DeMattos R B, Bales K R, Cummins D J, Dodart J C, Paul S M, Holtzman D M. 2001. Peripheral anti-A beta antibody alters CNS and plasma A beta clearance and decreases brain A beta burden in a mouse model of Alzheimer's disease. Proc. Natl. Acad. Sci. U.S.A. 98: 8850-8855. 10.1073/pnas.151261398 [doi]; 151261398 [pii].
4. Donohue M C, Moghadam S H, Roe A D, Sun C K, Edland S D, Thomas R G, Petersen R C, Sano M, Galasko D, Aisen P S, Rissman R A. 2014. Longitudinal plasma amyloid beta in Alzheimer's disease clinical trials. Alzheimers Dement. S1552-5260(14)02769-1 [pii].
5. Hartmann T et al. (September 1997). “Distinct sites of intracellular production for Alzheimer's disease A beta40/42 amyloid peptides”. Nat. Med. 3 (9): 1016-20.
6. Hoist J J. 2007. The Physiology of Glucagon-like Peptide 1. Physiol. Rev. 87: 1409-1439. 10.1152/physrev.00034.2006.
7. Kulstad J J, Green P S, Cook D G, Watson G S, Reger M A, Baker L D, Plymate S R, Asthana S, Rhoads K, Mehta P D, Craft S. 2006a. Differential modulation of plasma beta-amyloid by insulin in patients with Alzheimer disease. Neurology 66: 1506-1510. 66/10/1506 [pii]; 10.1212/01.wnl.0000216274.58185.09 [doi].
8. Kulstad J J, Savard C E, Lee S P, Craft S, Cook D G. 2006b. P2-020: Liver-mediated clearance of peripheral amyloid-beta (1-40). Alzheimer's and Dementia, 2: S237-S238.
9. Meier J J, Holst J J, Schmidt W E, Nauck M A. 2007. Reduction of hepatic insulin clearance after oral glucose ingestion is not mediated by glucagon-like peptide 1 or gastric inhibitory polypeptide in humans. Am. J. Physiol. Endocrinol. Metab. 293: E849-56. 10.1152/ajpendo.00289.2007.
10. Oh E S, Troncoso J C, Fangmark Tucker S M. 2008. Maximizing the Potential of Plasma Amyloid-Beta as a Diagnostic Biomarker for Alzheimer's Disease. Neuromolecular Med. 10.1007/s12017-008-8035-0.
11. Oh E S, Mielke M M, Rosenberg P B, Jain A, Fedarko N S, Lyketsos C G, Mehta P D. 2010. Comparison of conventional ELISA with electrochemiluminescence technology for detection of amyloid-beta in plasma. J. Alzheimers Dis. 21: 769-773. 10.3233/JAD-2010-100456.
12. Perry T, Lahiri D K, Sambamurti K, Chen D, Mattson M P, Egan J M, Greig N H. 2003. Glucagon-like peptide-1 decreases endogenous amyloid-beta peptide (Abeta) levels and protects hippocampal neurons from death induced by Abeta and iron. J. Neurosci. Res. 72: 603-612. 10.1002/jnr.10611.
13. Ritchie C, Smailagic N, Noel-Storr A H, Takwoingi Y, Flicker L, Mason S E, McShane R. 2014. Plasma and cerebrospinal fluid amyloid beta for the diagnosis of Alzheimer's disease dementia and other dementias in people with mild cognitive impairment (MCI). Cochrane Database Syst. Rev. 6: CD008782. 10.1002/14651858.CD008782.pub4 [doi].
14. Selkoe D J. 1999. Translating cell biology into therapeutic advances in Alzheimer's disease. Nature 399: A23-31.
15. Sperling R A, Jack C R, Jr, Aisen P S. 2011. Testing the right target and right drug at the right stage. Sci. Transl. Med. 3: 111 cm33. 10.1126/scitranslmed.3002609 [doi].
16. Takeda S, Sato N, Uchio-Yamada K, Yu H, Moriguchi A, Rakugi H, Morishita R. 2012. Oral glucose loading modulates plasma beta-amyloid level in alzheimer's disease patients: potential diagnostic method for Alzheimer's disease. Dement. Geriatr. Cogn. Disord. 34: 25-30. 10.1159/000338704 [doi].
17. Takeda S, Sato N, Uchio-Yamada K, Sawada K, Kunieda T, Takeuchi D, Kurinami H, Shinohara M, Rakugi H, Morishita R. 2009. Elevation of plasma beta-amyloid level by glucose loading in Alzheimer mouse models. Biochem. Biophys. Res. Commun. 385: 193-197. 10.1016/j.bbrc.2009.05.037.
18. Watson G S, Peskind E R, Asthana S, Purganan K, Wait C, Chapman D, Schwartz M W, Plymate S, Craft S. 2003. Insulin increases CSF Abeta42 levels in normal older adults. Neurology 60: 1899-1903.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention may become apparent to those skilled in the art upon review of this specification. The full scope of the invention should be determined by reference to the claims, along with their full scope of equivalents, and the specification, along with such variations. Such equivalents are intended to be encompassed by the following claims.

1. A method of identifying a mammal having a neurological disease or disorder, or at risk for developing a neurological disease or disorder, comprising:
a) obtaining a biological sample from the mammal;
b) subjecting the mammal to a glucose tolerance test (GTT);
c) obtaining a biological sample from the mammal after GTT;
d) determining the level of expression or level of activity, or change in level of expression or activity before and after GTT of one or more biomarkers selected from Aβ 40, Aβ 42, and both, in the mammal sample;
e) determining the level of expression or level of activity, or change in level of expression or activity before and after GTT of said one or more biomarkers in a standard control; and
f) comparing the level of expression or level of activity, or change in level of expression or activity before and after GTT of said one or more biomarkers determined in steps c) and d);
wherein modulation in the level of expression or level of activity, or change in level of expression or activity before and after GTT of the one or more biomarkers in the mammal sample relative to the standard control level of expression or level of activity, or change in level of expression or activity before and after GTT of the one or more biomarkers indicates that the mammal is afflicted with a neurological disorder, or at risk of developing a neurological disorder.

2. The method of claim 1, wherein the glucose tolerance test is oral.

3. The method of claim 1, wherein the mammal is a human.

4. The method of claim 1, wherein the neurological disease or disorder is mild cognitive impairment (MCI) or Alzheimer's disease (AD).

5. The method of claim 1, wherein the modulation is calculated as a change (Δ) in plasma Aβ levels.

6. The method of claim 5, wherein the change (Δ) in plasma Aβ is calculated as the higher level of plasma Aβ from either 0 (baseline) or about 5 minutes after ingestion of oral glucose solution to the about 10 minute time point after ingestion.

7. The method of claim 5, wherein the modulation is an increase or no Δ in Aβ 40, Aβ 42, or both, in said mammal after OGTT when compared to control.

8. The method of claim 7, wherein the modulation is aincrease ranging from a Δ Aβ 40 of about −140 pg/ml to about 60 pg/ml in the patient versus a Δ Aβ 40 of about −35 pg/ml to about 270 pg/ml in the control subject and a Δ Aβ 42 of about −15 pg/ml to about 6 pg/ml in the patient versus a Δ Aβ 42 of about −2 pg/ml to about 60 pg/ml in the control subject.

9. The method of claim 8, wherein the modulation is a increase ranging from a Δ Aβ 40 of about −135.66 pg/ml to about 58.43 pg/ml in the patient versus a Δ Aβ 40 of about −32.15 pg/ml to about 263.51 pg/ml in the control subject and a Δ Aβ 42 of about −11.63 pg/ml to about 5.80 pg/ml in the patient versus a Δ Aβ 42 of about −1.38 pg/ml to about 57.21 pg/ml in the control subject.

10-30. (canceled)

31. A method for monitoring the progression of a neurological disease or disorder in a patient or monitoring the effectiveness of a therapeutic agent or treatment of a patient having a neurological disease or disorder, the method comprising:
(i) obtaining a pre-administration sample from a patient prior to administration of the therapeutic agent or treatment;
(ii) subjecting the patient to OGTT;
(iii) detecting the levels of one or more biomarkers in the pre-administration sample; (iv) obtaining one or more post-administration samples from the patient;
(v) detecting the change in the levels of one or more biomarkers in the post-administration samples;
(vi) comparing the level of one of more biomarkers in step (iii) pre-administration sample with the level of one of more biomarkers in step (v) in the post-administration sample or samples; and
(vii) altering the treatment of the patient.

32. The method of claim 31, wherein the neurological disease or disorder is mild cognitive impairment (MCI), Alzheimer's disease (AD), or both.

33. The method of claim 31, wherein between the first point in time (i) and the subsequent point in time (iv), the patient has undergone treatment, completed treatment, and/or is in remission for the neurological disease or disorder.

34. The method of claim 31, wherein the one or more biomarkers is selected from Aβ 40, Aβ 42, insulin, GLP-1, and any combination thereof.

35. The method of claim 34, wherein the one or more biomarkers are Aβ 40 and Aβ 42.

36. The method of claim 31, wherein in step (iv), the comparison yields a change (Δ) in plasma Aβ40 and Aβ 42 levels.

37. The method of claim 36, wherein the change (Δ) in plasma Aβ is calculated as the higher level of plasma Aβ from either 0 (baseline) or about 5 minutes after ingestion of oral glucose solution to the 10 minute time point after ingestion.

38. The method of claim 36, wherein the A is an increase or no Δ in Aβ 40, Aβ 42, or both, in said patient in (iii) and (v).

39. The method of claim 38, wherein in the increase or no Δ in Aβ 40, Aβ 42, or both, indicates that the treatment or therapeutic agent is ineffective.

40. The method of claim 32, wherein the amount or dose of the therapeutic agent against MCI/AD is increased.

41. The method of claim 36, wherein in the increase or no Δ in Aβ 40, Aβ 42, or both, indicates that the treatment or therapeutic agent is preventing or delaying the progression of the neurological disease or disorder or symptom thereof.
